(19) United States
(12) Patent Application Publication
(10) Pub. No.: US 2023/0309975 A1
(43) Pub. Date: Oct. 5, 2023

(54) TONGUE DEPRESSOR AND ORAL/NASAL SWAB MOUNTED SHIELD, METHOD OF USE, AND METHOD OF MANUFACTURE

(71) Applicant: Groman Inc., Margate, FL (US)

(72) Inventor: Boaz Barry Groman, Boca Raton, FL (US)

(21) Appl. No.: 18/129,833

(22) Filed: Apr. 1, 2023

Related U.S. Application Data
(60) Provisional application No. 63/326,275, filed on Apr. 1, 2022.

(51) Int. Cl. A61B 13/00

(52) U.S. Cl. CPC A61B 13/00

(57) ABSTRACT

A PPE shield mounted on a tongue deflector (depressor) or an oral/nasal swab establishes a barrier between the patient and a healthcare provider to substantially block aerosols and the like which may be exhaled or expelled by the patient, thereby protecting the healthcare provider from exposure to these aerosols and the like while performing a procedure. The shield may be generally concave, such as frustroconical or hemispherical. The depressor or swab may be inserted through an opening in the shield. A support structure may be formed at or around the opening. A flat view port may be provided in the shield, allowing the healthcare provider to have an unobstructed and undistorted view of the patient's oral cavity or nasal passage while performing the procedure. The shield may be vacuum formed.

### CROSS-REFERENCE TO RELATED APPLICATIONS

This is a nonprovisional filing of 63/326,275 filed 1 Apr. 2022.

### FIELD OF THE INVENTION

The invention disclosed herein relates to providing a system for improved protection from contagious diseases. More particularly the invention relates to providing a system for protection a medical practitioner during oral or nasal examination of a patient. Patients having a contagious disease may transmit that disease to the medical practitioner through body fluids expelled from the mouth or nose, during an oral or nasal examination. The medical practitioner needs to be close to the patient to examine and take culture swabs from the patient's mouth/throat or nose. It is desirable, however, to prevent potential spread of disease to the medical practitioner.

### BACKGROUND

In the 2020 Coronavirus pandemic, treatment options for patients with shortness of breath were limited due to the fear that first line and hospital personnel would be exposed to aerosolized viral particles expelled by patients into the environment. Prior to the pandemic, aerosolized medication inhaled by healthcare providers has also been an issue which this invention also addresses.

The invention disclosed herein relates primarily to providing a “first line of defense” for a healthcare provider from pathogens and particles which may be expelled from a patient's mouth or nose. Essentially, the invention relates to a shield which may be used in conjunction with performing an examination of a patient's oral cavity, particularly when using a tongue depressor.

Additional means, such as PPE (personal protective equipment) may be used (worn) by the healthcare provider to provide additional defense. PPE is the “last line of defense” that a healthcare provider has to protect themselves against infectious disease. Generally, PPE is only there to protect the healthcare provider if, in a rare instance, all other lines of defense against infection have failed.

The term PPE refers to protective clothing, including gloves, face shields, eyewear, facemasks, and/or other equipment designed to protect the wearer from injury or the spread of infection. Most commonly used in health-care settings, PPE acts as a barrier between infectious materials and your skin, mouth, nose, or eyes. It is essential, however, that PPE is used with other infection control practices such as hand washing, using alcohol-based hand sanitizers, and the consistent enforcement of infection prevention practices.

The invention disclosed herein may be used in conjunction with other precautions and following protocols that promote safe environments for the healthcare provider.

U.S. Pat. No. 9,895,141 (20 Feb. 2018; Schultz), entitled Contagion Prevention Systems, discloses a device for preventing the spread of bio-matter contagions when used in conjunction with a tongue depressor. The device includes a shield for intercepting the bio-matter contagions. The shield adjustably and removably secures along the tongue depressor. The device also includes a grip manipulator that extends from the shield. The grip manipulator adjustably engages the tongue depressor. The device also includes a window that provides viewing access through the shield.

U.S. Pat. No. 8,814,897 (26 Aug. 2014; Schultz), entitled Contagion Prevention Systems, discloses a system for improved protection from contagious diseases. More particularly this invention relates to providing a system for protection a medical practitioner during oral examination of a patient. Patients having a contagious disease may transmit that disease to the medical practitioner through body fluids expelled from the mouth, during an oral examination. The medical practitioner needs to be close to the patient to orally examine and take culture swabs from the patient's mouth and throat. It is desirable, however, to prevent potential spread of disease to the medical practitioner.

### RELATED REFERENCES (PUBLICATIONS)

Some other US patent publications which may be relevant to the invention(s) disclosed herein may include:

| | | |
|---|---|---|
| 2,549,514 | April 1951 | Oertel |
| 3,537,447 | November 1970 | Gauthier et |
| 3,545,433 | December 1970 | Horn |
| 4,275,719 | Jun. 30, 1981 | Mayer |
| 4,697,578 | Oct. 6, 1987 | Burgin |
| 4,958,623 | Sep. 25, 1990 | Rocco |
| 5,360,018 | Nov. 1, 1994 | Chen |
| 5,562,686 | Oct. 8, 1996 | Sauer et al. |
| 20020108614 | Aug. 15, 2002 | Schultz |
| 20080242941 | Oct. 2, 2008 | Kim |

### SUMMARY

It is a general overall object of the invention to provide protection for a healthcare provider (such as a doctor) from aerosols and the like which may be exhaled or expelled by a patient during an oral or nasal exam, while the healthcare provider is using a tongue depressor or nasal swab, respectively.

In the main, hereinafter, using a tongue depressor for an oral examination may be discussed. The invention is applicable to nasal examinations.

Other (different, additional) applications (uses) may be within the scope of the invention.

According to the invention, generally, a PPE (personal protective equipment) shield is provided for use by healthcare provider performing a procedure on a patient. The shield may be formed of a plastic sheet, and may be cup-shaped, semi-spherical (hemispherical), or otherwise generally concave. The concavity will be oriented towards the patient. An orifice (opening) is provided in the shield through which an instrument such as a tongue depressor or nasal swab may be inserted and supported, and ultimately manipulated by a healthcare provider providing the procedure on a patient.

The concave shield may be in the form of a truncated cone (or “frustrum”, or frustroconical), rather than semi-spherical (hemispherical), with a flat area forming a view port located at the (flat) top surface of the truncated cone.

A semi-spherical shield may have a diameter (width) of approximately 10 cm (4 inches). A shield in the form of a truncated cone may have a base diameter of approximately 10 cm (4 inches).

The shield may be alternatively be flat, although shields which are curved (dish-shaped or concave) may provide better conformity to the generally convex shape of a patient's face and provide commensurately better protection for the healthcare provider.

The shield may be fabricated from a thin, such as 0.005″-0.020″ (0.1 mm-0.5 mm) thick plastic material such as PET (polyethylene terephthalate).

The shield may be relatively rigid—i.e., able to maintain its shape when being handled by the healthcare provider. In other words, the shield should not deform when slight forces (such as less than 1 psi) are applied thereto, such as when inserting a tongue depressor or nasal swab.

The plastic material may be substantially transparent, to allow the healthcare provider to view the patient's oral cavity and/or nasal passage(s).

The shield may be sized (and shaped) to fit over (in front of) the mouth of a patient, including the patient's jaw. A semi-spherical shield may have a diameter (width) of approximately 10 cm (4 inches).

The shield may be sized (and shaped) to additionally fit over (in front of) the patient's nose.

The shield may have a shape other than semi-spherical, such as frustroconical, having a width at its base of approximately 10 cm (4 inches) and a height (depth or radius, if semi-spherical) of approximately 5 cm (2 inches). In either case, the shield is “dish-shaped”.

The shield is intended for use with an instrumentality such as a tongue depressor or swab, or the like. The instrumentality itself would be provided separately, and is not an element of the invention.

The shield may include a support structure (or element, such as a “lip” or protruding rim”) disposed on an inner or outer surface of the shield, disposed at the location of (surrounding) the opening for the tongue depressor or swab. The support structure may also be referred to as a “retaining feature”. (See. e.g., APPENDIX, page 13) The purpose of the support structure (retaining feature) is to provide stability (some resistance to being moved) to an instrumentality (depressor or swab) which is inserted though the opening in the shield, since the shield is generally too thin to provide much stability, while allowing the depressor or swab to be manipulated by the healthcare provider, such as by moving the depressor or swab in (towards the patient) or out (away from the patient) of the opening in the shield, including manipulating the angle of the depressor or swab whilst it is retained in the opening of the shield.

The support structure may simply be a region (such as donut shaped) disposed at or around (surrounding, or partially surrounding) the opening which is thicker than remaining portions of the shield (i.e., greater than the thickness of the sheet forming the shield). In other words, the support structure may be a protruding portion of the shield, around or adjacent to the opening. The support structure may completely or only partially surround the opening.

The support structure may be disposed on an external (towards the healthcare provider) surface of the shield. The support structure may alternatively be disposed on the internal (towards the patient's face) surface of the shield. The support structure may comprise a region (surrounding the opening) of increased thickness on both the internal and external surfaces of the shield. The support structure may be integrally formed with the shield (i.e., the support structure is not a separate element assembled to the shield.) Page 3 of the APPENDIX provides some examples of an external support structure (upper illustration) and an internal support structure (lower illustration).

Generally, a tongue depressor, which may have a uniform profile and thickness along its length, may be inserted through the opening in the shield from either the inner (internal, towards the patient) or outer (external, towards the healthcare provider) surface thereof. However, it may be necessary to insert a swab, which has an enlarged portion at one end thereof, through the opening in the shield, by the end which does not have the enlarged portion.

When placed onto (in front of) the patient's face, the shield establishes a barrier between the patient and the healthcare provider to substantially block aerosols and the like which may be exhaled or expelled by the patient, thereby protecting the healthcare provider from exposure to these aerosols and the like.

The shield may be fabricated as a single element.

In use, an internal (interior, inner) surface of the shield is oriented towards the patient's face. An external (exterior, outer) surface of the shield is oriented towards the healthcare provider.

The shield may have an orifice (opening), which may be in the form of a slot or slit which is sized to receive a tongue depressor or nasal swab with a slight interference fit which allows the tongue depressor or nasal swab to be inserted through the shield, resulting in the shield being supported on the tongue depressor or nasal swab during performing an oral or nasal procedure, respectively. In other words, the orifice may be only slightly, such as a few thousandths of an inch smaller than the cross-section of the tongue depressor or nasal swab.

The shield may be provided with a tubular extension element extending from the orifice, to provide additional stability to the tongue depressor or nasal swab, when it is inserted through the shield. The tubular extension element provides more stability to the shield when mounted on the tongue depressor or nasal swab.

The tubular extension element may be formed integrally with the shield, and may extend either internally (towards the patient) or externally (towards the healthcare provider) from the shield.

The tubular extension element may be referred to as a support or retaining or mounting feature, and may extend a few millimeters from the internal or external surface of the shield.

A portion of the shield may be flat, to provide a “view port” (viewing area) for the healthcare provider so that the healthcare provider may enjoy an unobstructed and undistorted view of the patient's oral cavity or nasal passage while performing the procedure. This may be important because the generally convex shield has curved surfaces.

A preferred embodiment may have a larger flat area to provide a larger viewing area into the patient's mouth (reducing light deformation due to curvature of the shield).

The tongue depressor (or oral swab) mounting feature (i.e., orifice and tubular extension element) may be centered on the shield or viewing area thereof.

The tongue depressor (or oral swab) mounting feature (i.e., orifice and tubular extension element) may be offset (“off-center”) from the center of the shield or viewing area thereof to provide additional visibility, for example at the top side of the Tongue Depressor.

In use, the tongue depressor or oral swab is inserted through the tubular extension element of the shield, with an interior portion of the tongue depressor or oral swab extending beyond the shield to be inserted in the patient's oral or nasal cavity, and an exterior portion of the tongue depressor or oral swab extending beyond the shield to be manipulated by the healthcare provider.

The shield may be used without mounting or temporarily securing the shield onto the patient's face.

The novelty of the shield may be the way that it slides onto the Tongue Depressor and retains its structure. It is intended that the shield is securely mounted on the tongue depressor (or swab) which has been inserted through the opening and corresponding support feature.

The shield may be a simple vacuum molded device such as a clear PET (polyethylene terephthalate) domed cup cover. See https://www.webstaurantstore.com/choice-9-oz-12-oz-16-oz-20-oz-24-oz-clear-pet-dome-lid-with-no-hole-case/500LDOMENH.html.

The shield may cost only a few cents (<$0.02 in mass production) and would be a low cost solution to reducing contamination due to patient gag reflex cough.

The shield may be formed, and mass produced inexpensively, by a vacuum forming process.

The shield is intended for single use (disposable).

Other objects, features and advantages of the invention(s) disclosed herein may become apparent in light of the following illustrations and descriptions thereof.

### BRIEF DESCRIPTION OF THE DRAWINGS

Reference will be made in detail to embodiments of the disclosure, non-limiting examples of which may be illustrated in the accompanying drawing figures (FIGS.). The figures may generally be in the form of diagrams. Some elements in the figures may be stylized, simplified or exaggerated, others may be omitted, for illustrative clarity.

Although the invention is generally described in the context of various exemplary embodiments, it should be understood that it is not intended to limit the invention to these particular embodiments, and individual features of various embodiments may be combined with one another. Any text (legends, notes, reference numerals and the like) appearing on the drawings are incorporated by reference herein.

FIG. 1 is a diagram (perspective view) of a tongue deflector version of a shield with an external support feature disposed off-center on the view port, according to an embodiment of the invention.

FIG. 2 is a diagram (perspective view) of a swab version of a shield with an external support feature disposed off-center on the view port, according to an embodiment of the invention.

Some additional figures (and text) are presented in the APPENDIX, and are incorporated by reference herein.

### DESCRIPTION

Various embodiments (or examples) may be described to illustrate teachings of the invention(s), and should be construed as illustrative rather than limiting. It should be understood that it is not intended to limit the invention(s) to these particular embodiments. It should be understood that some individual features of various embodiments may be combined in different ways than shown, with one another. Reference herein to “one embodiment”, “an embodiment”, or similar formulations, may mean that a particular feature, structure, operation, or characteristic described in connection with the embodiment is included in at least one embodiment of the present invention. Some embodiments may not be explicitly designated as such (“an embodiment”).

The invention disclosed herein is generally a shield, which may be referred to as a “PPE shield” which is intended to be mounted on a tongue depressor or oral/nasal swab, so as to provide a barrier between healthcare provider and patient.

Patients have the tendency to cough and sneeze when probed with a Tongue Depressor and Oral/Nasal Swab. Coughing and sneezing projectiles patient aerosol out of the oral and/or nasal cavity and onto the healthcare provider. Some of the aerosol is in fine particles which are suspended in the air but most of the aerosol is in larger particles which are carried through the air to settle wherever they impact on the healthcare provider and surroundings. This requires the healthcare provider to wear PPE and substantial disinfection of the environment post procedure.

The innovation disclosed herein is a “shield” that provides a simple and low-cost PPE solution to capturing the larger aerosol particles thereby providing additional protection to healthcare providers.

The shield mounts onto Tongue Depressor or Oral/Nasal Swab and provides a clear view of the patient's anatomy (mouth or nasal cavity) so the procedure can be performed as usual.

During performing the procedure, the shield, with tongue depressor or swab mounted therein, is positioned by the healthcare provider in front of the patient's mouth and nose to block aerosol discharge so that it does not transfer to the healthcare provider.

Because cost is always a consideration with PPEs as single use devices, it is imperative to implement a low cost material and fabrication technology.

The shield may readily be manufactured utilizing vacuum forming technology such as is used to manufacture single use cup lids and disposable food packages.

The shield may be formed of thin, inexpensive material (such as, but not limited to PET) that may quickly and easily be formed to shape. The material may also be available in clear transparent materials.

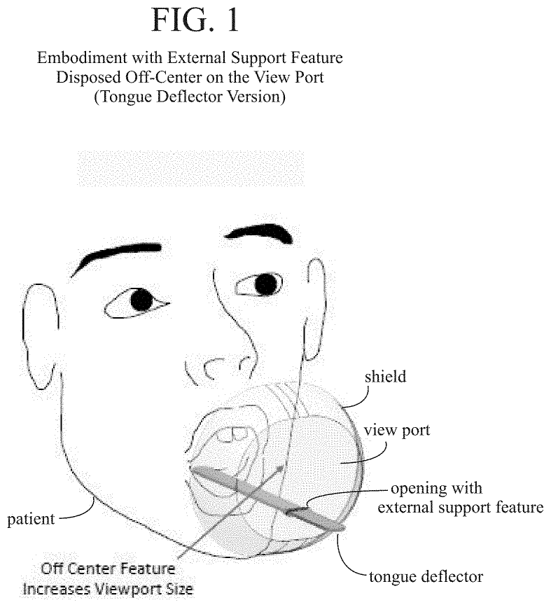

FIG. 1 is a diagram (perspective view) of a tongue deflector version of a shield with an external support feature disposed off-center on the view port, according to an embodiment of the invention. This figure demonstrates the shield acting as a barrier between the healthcare provider side of the tongue deflector and the patient's oral and nasal cavities.

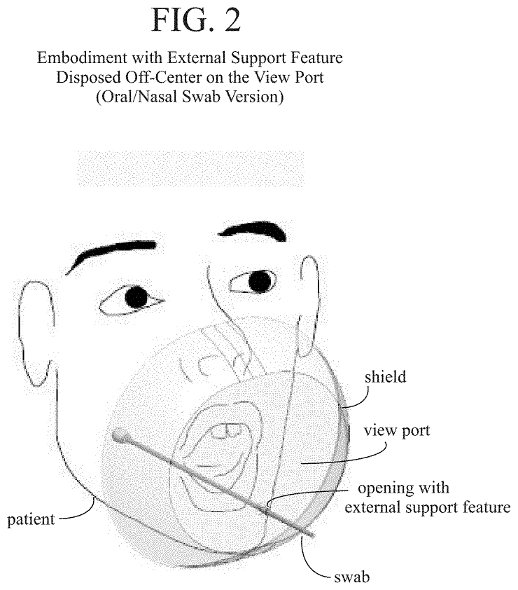

FIG. 2 is a diagram (perspective view) of a swab version of a shield with an external support feature disposed off-center on the view port, according to an embodiment of the invention. This figure demonstrates the shield acting as a barrier between the healthcare provider side of the swab and the patient's oral and nasal cavities.

### APPENDIX

Appended hereto and forming part of the disclosure hereof is an 18 page document entitled “Tongue Deflector Shield”. The Appendix describes the innovation process as an Engineering Notebook would, in chronological order. Therefore, the embodiments disclosed in the last few pages of the Appendix may be preferred. As disclosed therein:

Page 1 shows a perspective view (left) and a side view (right) of a simple shield with a tongue depressor inserted therethrough.

Page 2 shows that the shield may not be well supported on the tongue depressor due to the very limited area of contact between the tongue depressor and the shield.

Page 3 shows that a support feature may be incorporated into (integrally formed with) the shield to provide stability to the shield when mounted on a tongue depressor. The tongue depressor extends through an orifice (opening, slit or slot) in the shield. The support feature is generally a tubular extension of the shield, extending from the orifice, and provides additional support and stability for the tongue depressor mounted shield.

As shown in the upper figure, the support feature may extend towards the outside of the shield (towards the healthcare provider).

As shown in the lower figure, the support feature may extend towards the inside of the shield (towards the patient).

The support feature is sized and shaped to securely retain the shield on the tongue depressor.

Page 4 shows the shield mounted on a tongue depressor in use with a patient. The two figures illustrate that a portion of the shield may be formed as a flat view port or window to provide the healthcare provider with an undistorted view of the patient's anatomy.

Page 5 shows in greater detail (three views) the shield with the support feature extending internally from the shield, towards the patient. The support feature for a tongue depressor may be a flat, tubular element, extending a few millimeters from the internal surface of the shield.

Page 6 shows (three views) that a portion of the shield may be flat, providing a view port.

The figure on the right shows that stiffening features (ribs) may be incorporated into the surface of the shield to make an otherwise flexible shield stiffer, without increasing material thickness.

The figure on the right also shows that there is a friction fit between the tongue depressor and shield which is sufficient to prevent the shield from sliding off of the tongue depressor, during use.

Page 7 shows a prototype vacuum forming mold used to produce an embodiment of the shield for use with tongue depressors.

In this embodiment, the support feature extends towards the interior of the shield. A flat area for the view port is evident.

Channels for forming the stiffening features (ribs) are visible on the mold.

Page 8 shows (four views) a closeup or the fabrication steps, for an embodiment with the support feature (or simply “feature”) extending inwardly (patient side) from the shield.
Step 1: the support feature, projecting from the inner surface of the shield, is vacuum formed
Step 2: the tip (inner end) of the feature is excised, cut-off, removed
Step 3: the feature is now clear (open) so that the tongue deflector can be inserted therethrough
Step 4: the tongue deflector is inserted through the feature, either from the inside or the outside

Page 9 shows (four views) a closeup or the fabrication steps, for an embodiment with the support feature (or simply “feature”) extending inwardly (patient side) from the shield.
Step 1: the support feature, projecting from the inner surface of the shield, is vacuum formed
Step 2: a slit is formed in the tip (inner end) of the feature
Step 3: the feature is ready to accept the tongue deflector (or may be further cut for easier insertion of the tongue deflector.)
Step 4: the tongue deflector is inserted through the feature, either from the inside or the outside Note: In this embodiment, no material need be removed (contrast the embodiment on Page 8)

Page 10 shows (two views) of the tongue deflector with internal retaining/support feature disposed off-center in the flat viewing area (view port) to allow better visibility of the area being treated (such as the patient's oral cavity)
note: the left illustration corresponds with FIG. 1. The internally-extending feature is not very visible in this view. See detail of internally-extending feature on Pages 8, 9.

Page 11 shows (two views) of an embodiment similar to the embodiment shown on Page 10, but with the retaining/support feature extending external from the flat area (view port) of the shield. The external feature is shown off-center similar to the Page 10 embodiment.
note: The feature, whether internal or external, may be disposed on the shield at other than the flat view port area of the shield.
note: The feature, whether internal or external, may extend only a few millimeters from the surface of the shield (or view port)

Page 12 shows (four views) a closeup or the fabrication steps, for an embodiment with the support feature (or simply “feature”) extending externally (caregiver side) from the shield.
Step 1: the support feature, projecting from the inner surface of the shield, is vacuum formed
Step 2: the tip (inner end) of the feature is excised, cut-off, removed
Step 3: (internal view) the feature is now clear (open) so that the tongue deflector can be inserted therethrough
Step 4: (external view) the tongue deflector is inserted through the feature, either from the inside or the outside

Page 13 shows (two views, plus detail) of an embodiment with the support feature extending externally from the shield. The closeup view shows the slit for receiving the tongue depressor (or deflector).
note: in these figures, the shield is shown white, rather than transparent, for illustrative clarity

Page 14 shows (three views) of an embodiment with the support feature extending externally from the shield. The tongue depressor/deflector is shown extending through the support feature.
note: in these figures, the shield is shown white, rather than transparent, for illustrative clarity
note: In this embodiment, no material need be removed (contrast the embodiment on Page 12)
NOTE: It may be observed, in several of the embodiments illustrated herein, that the shield is in the form of a truncated cone (or “frustrum”), with the view port at the top surface of the truncated cone. This has the advantage that the view port is on a flat surface. The side surfaces of the shield may be substantially straight, rather than curved in the semi-spherical versions of the shield.

Page 15 shows (two views) of an embodiment with the support feature extending externally from the shield. The tongue depressor/deflector is shown extending through the support feature.
note: in these figures, the shield is shown white, rather than transparent, for illustrative clarity
note: in the left hand figure, the stiffening ribs (or channels) in the shield are visible
note: the closeup of the support feature shows the expansion (spreading) of the support feature over the tongue depressor to create an interference fit which provides stability to the shield and inhibits (or prevents) unintended sliding along the tongue depressor during the procedure.

Page 16 shows (one view) of a prototype vacuum forming mold used to produce an embodiment of the shield with a externally-extending support feature (for a tongue depressor).

Page 17 shows (two views) an embodiment of the shield intended for use with an oral/nasal swab. Compare FIG. 2.

This version is shown being used with a swab. The opening for the swab is disposed off center on the view port (top surface of the frustrum).

In the left hand view, the shield is oriented (with respect to the patient) so that the opening is disposed below the center of the shield (i.e., view port), to allow access to the patient's oral cavity and an unobstructed view of the oral cavity by the caregiver.

In the right hand view, the shield is oriented (with respect to the patient) so that the opening is disposed above the center of the shield (i.e., view port), to allow access to the patient's nasal cavity and an unobstructed view of the nasal cavity by the caregiver.

Page 18 shows (two views) an embodiment of the shield intended for use with an oral/nasal swab.

NOTE: Design considerations for the tongue deflector version of the shield are also applicable to the swab version of the shield, the main difference being in the form factor of the support element extending from the internal or external surface of the shield.

Regarding Internal v. External Feature From the vacuum forming process perspective, the external features are easier to achieve because the vacuum has more surface to act upon as the warm material is ‘lowered’ over the mold. That generates higher forces on the warm material which permits the fabrication of more intricate features (see Appendix; Page 16).

Internal features formation is limited by their surface area and therefore less force is available for their creation. Referring to Page 7 of the Appendix, as the warm material is ‘lowered’ over the mold, once the warm material makes contact with the flat surface the vacuum force is limited to the feature opening.

### DISCLAIMER

It is important to note that while shields and covers can provide some protection from bodily fluids, they are not foolproof and healthcare providers should still take precautions to minimize their risk of exposure. This can include wearing gloves, masks, and eye protection, as well as following proper hand hygiene practices.

While the invention(s) may have been described with respect to a limited number of embodiments, these should not be construed as limitations on the scope of the invention(s), but rather as examples of some of the embodiments of the invention(s). Those skilled in the art may envision other possible variations, modifications, and implementations that are also within the scope of the invention(s), and claims, based on the disclosure(s) set forth herein.

What is claimed is:

1. A PPE shield for use by a healthcare provider when performing an oral or nasal procedure on a patient, said shield comprising:
a sheet of plastic material;
an opening in the plastic material; and
a support element surrounding the opening and extending from at least one of the inner or outer surfaces of the shield.

2. The PPE shield of claim 1, wherein:
the sheet of material is concave, to conform to the generally convex shape of the patient's face.

3. The PPE shield of claim 1, wherein:
the sheet of material is in the shape of a truncated cone (frustrum) or hemispherical.

4. The PPE shield of claim 1, wherein:
the plastic material is substantially transparent.

5. The PPE shield of claim 1, wherein:
the plastic material comprises PET (polyethylene terephthalate).

6. The PPE shield of claim 1, wherein:
the shield is sufficiently rigid to not deform when slight forces are applied thereto, such as when inserting a tongue depressor or nasal swab through the opening, and subsequently manipulating the depressor or swab.

7. The PPE shield of claim 1, wherein:
the shield is sized and shaped to fit over (in front of) the mouth of a patient, including the patient's jaw.

8. The PPE shield of claim 1, wherein:
the shield is sized and shaped to fit over (in front of) the lower facial area of a patient, including patient's mouth and optionally the patient's nose.

9. The PPE shield of claim 1, wherein:
in use, when the shield placed onto (in front of) the patient's face, the shield establishes a barrier between the patient and a healthcare provider to substantially block aerosols and the like which may be exhaled or expelled by the patient, thereby protecting the healthcare provider from exposure to these aerosols and the like.

10. The PPE shield of claim 1, wherein:
the opening is in the form of a slot or slit which is sized to receive a tongue depressor or nasal swab with a slight interference fit which allows the tongue depressor or nasal swab to be inserted through the shield, resulting in the shield being supported on the tongue depressor or nasal swab during performing the oral or nasal procedure, respectively.

11. The PPE shield of claim 1, further comprising:
a support structure disposed at or around the opening to provide stability to a tongue depressor or nasal swab inserted through the opening.

12. The PPE shield of claim 1, wherein:
the support structure is formed integrally with the shield, and extends either internally (in use, towards the patient) or externally (in use, towards the healthcare provider) from a respective inner or outer surface of the shield.

13. The PPE shield of claim 11, wherein:
the support structure is in the form of a tubular extension of the opening.

14. The PPE shield of claim 1, wherein:
a portion of the shield is substantially flat, providing a view port allowing the healthcare provider to have an unobstructed and undistorted view of the patient's oral cavity or nasal passage while performing the procedure.

15. The PPE shield of claim 14, wherein:
the opening is disposed on the view port.

16. The PPE shield of claim 14, wherein:
the opening is disposed offset from a center of the view port.

17. The PPE shield of claim 1, wherein:
the opening is disposed offset from a center of the overall shield.

18. Method of performing an oral or nasal procedure on a patient, comprising:
providing a shield as described in claim 1;
inserting a tongue depressor or swab through the opening of the shield; and
performing the procedure.

19. Method of manufacturing a PPE shield, comprising:
vacuum forming the shield of claim 1, including a protruding portion of the shield which forms a support structure; and
then cutting or slitting the protruding portion of the shield which forms the support structure.
